(19) United States
(12) Patent Application Publication
(10) Pub. No.: US 2015/0217106 A1
(43) Pub. Date: Aug. 6, 2015

(54) Swab Devices

(71) Applicant: Excelsior Medical Corporation, Neptune, NJ (US)

(72) Inventors: Robert Banik, Edgewater, NJ (US); Christopher E. Gardner, Manalapan, NJ (US); Joshua M. Schmitz, Brick, NJ (US)

(73) Assignee: Excelsior Medical Corporation, Neptune, NJ (US)

(21) Appl. No.: 14/616,593

(22) Filed: Feb. 6, 2015

Related U.S. Application Data
(60) Provisional application No. 61/936,660, filed on Feb. 6, 2014.
(60) Provisional application No. 62/036,666, filed on Aug. 13, 2014.

(51) Int. Cl. A61M 39/16; A61M 39/20; A61M 39/02; A61L 2/18

(52) U.S. Cl. CPC A61M 39/162; A61L 2/18; A61M 39/16; A61M 39/20; A61M 39/02; A61F 13/36

(57) ABSTRACT

A device for cleaning access devices includes a body with a first opposing side, a second opposing side, and a closed end. The body defines a space for retaining a pad, such as an alcohol prep pad. The pad could contain an antiseptic, disinfectant, etc. The device could include a housing configured to engage an access device, such as a luer access device.

### RELATED APPLICATIONS

This application claims priority to U.S. Provisional Patent Application No. 61/936,660 filed on Feb. 6, 2014 and to U.S. Provisional Application No. 62/036,666 filed on Aug. 13, 2014, the entire disclosures of which are expressly incorporated herein by reference.

### BACKGROUND

1. Field

The present disclosure generally relates to the field of devices for cleaning and covering access devices.

2. Related Art

Medical implements, access devices, such as luer access devices, entry ports or needle ports, are generally disinfected or ‘prepped’ by utilizing an alcohol prep pad and wiping the surfaces for a prescribed time, or by using self-contained alcohol containing caps that screw on the luer devices or press onto ports. Both approaches disinfect the same area, but caps require less user input and have been demonstrated to be more effective at reducing the chance of infection.

### SUMMARY

Swab devices that are cost effective use the functionality of prep pads with structures to retain the disinfectant in contact with the luer access device without extensive user input. Advantageously, a less costly swab device has a form factor similar to the existing alcohol pads or utilizes existing and inexpensive alcohol pads as a design element. Less costly cap devices can also eliminate the absorbent pad entirely and retain the package and product together, eliminating the need for a removable lid stock.

In one aspect, a device for cleaning access devices includes a body with a first opposing side, a second opposing side, and a closed end. The body defines a space for retaining a disinfectant or pad, such as an alcohol prep pad. The pad could contain an antiseptic, disinfectant, etc. The device could include a housing configured to engage an access device, such as a luer access device.

In another aspect, a device for cleaning access devices includes a body with an upper surface and a lower surface. The body could include a disinfecting pad, the upper surface could include an annular ring and a central portion being pierceable by insertion of a luer access device. The lower surface could include a pouch.

In another aspect, an antiseptic cap for use with an access site is provided. The antiseptic cap includes a housing with a generally cylindrical sidewall defining a chamber having an open upper end, a rim formed at the open upper end, and an end wall closing an opposite end. A disinfectant is positioned within the chamber, and a punch through seal is applied at the rim.

### BRIEF DESCRIPTION OF THE DRAWINGS

The foregoing features will be apparent from the following Detailed Description, taken in connection with the accompanying drawings, in which:

FIG. 1 is a perspective view showing a prep pad and a cover device.

FIG. 2 is a side view of the cover device shown in FIG. 1.

FIG. 3 is a perspective view showing the cover device with the prep pad of FIG. 1 and a luer access device.

FIG. 4 is a view similar to that shown in FIG. 3, showing the flow of disinfectant after the prep pad has been pierced.

FIG. 5 is a perspective view of another aspect of the cover device.

FIG. 6 is a cross-sectional view of the cover device of FIG. 5.

FIG. 7 is a perspective view of a subassembly retained by the cover device shown in FIG. 5.

FIG. 8 is a perspective view of another aspect of the cover device and a prep pad;

FIG. 9 is a perspective view of the cover device shown in FIG. 8 in an open configuration;

FIG. 10 is a perspective view showing the cover device in a closed configuration and a luer access device.

FIG. 11 is a perspective view of another aspect of the cover device along with a prep pad.

FIG. 12 is a cross-sectional view of the cover device and the prep pad of FIG. 11.

FIG. 13 is a cross-sectional view of the cover device and the prep pad, showing the prep pad inserted into the cover device.

FIG. 14 is a perspective view showing the cover device with the prep pad and a luer access device.

FIG. 15 is a cross-sectional view showing another aspect of the cover device along with a prep pad and a luer access device;

FIG. 16 is another cross-sectional view of the cover device, the prep pad, and luer access device of FIG. 15.

FIG. 17 is a more detailed cross-sectional view of the cover device shown in FIG. 15;

FIG. 18 is a perspective view of another aspect of the cover device.

FIG. 19 is a rear view of the cover device shown in FIG. 18.

FIG. 20 is a perspective view of the cover device shown in FIG. 18.

FIG. 21 is a rear view of the cover device shown in FIG. 20.

FIG. 22 is a cross-sectional view of another aspect of the cover device.

FIG. 23 is a perspective view of the cover device of FIG. 22.

FIG. 24 is a perspective view of the cover device of FIG. 22 with a holder.

FIG. 25 is a cross-sectional view of the cap punch through seal shown in FIG. 23.

FIG. 26 shows a luer access device positioned to be pushed into the cover.

FIG. 27 shows a luer access device pushed into the cover.

FIG. 28 shows a luer access device fully seated in the cover.

FIG. 29 is a cross-sectional view of the luer access device and cover as shown in FIG. 27.

FIG. 30 is a more detailed view of the holder shown in FIG. 24.

FIG. 31 shows a cap with a cover and a flexible septum with an aperture that retains a disinfectant such as alcohol within a chamber formed by the inside of the cap and the septum.

FIG. 32 is a perspective view of the body shown in FIG. 31.

FIG. 33 is a perspective view of a cap with a recess that retains a punch through flap after valve insertion.

FIG. 34 is a perspective view of a cap with a punch through flap that remains attached to the cap after use.

### DETAILED DESCRIPTION

Devices for cleaning and covering luer access devices are disclosed. These devices can use existing alcohol swab pads/packages, or a thicker pad with more alcohol. Many embodiments use the concept of an alcohol prep pad augmented with a device that aids the user in accessing and displacing the alcohol within the package to apply the alcohol to the luer access device and to hold the luer access device in the alcohol. The devices may have the ability to be tethered and untethered by the user or during manufacturing of complementary luer access devices or similar devices and ports.

In one aspect, an alcohol packaged pad is used “as is” and is opened and placed in a wetted condition into a device that aids the user in swab placement, location, attachment and/or isolation to obtain a higher level of disinfecting. The device may include features that snap and hold it and the pad on the luer access device or similar device. The device could have an open face clam shell type design with a pocket for receiving the alcohol pad, the device capable of being folded over and snapped shut. A tether can clip onto the device being cleaned.

In another aspect, alcohol or disinfectant is accessed by piercing or punching holes in the foil wrapper and either wicking and/or squeezing the alcohol through a channel or sponge into a device having an appropriate geometry suited for enabling better disinfecting of a luer access device or similar device. The user does not need to open the package and remove the pad with this design. A device may come attached to pad, possibly with a pressure sensitive adhesive to attach the device to the outside of package. The pad could be trapped with serration teeth such that when the user pulls the device, it pierces the package foil. The device may also be separate from the pad such that the user slides the pad into the device or adheres the device to the pad package before use.

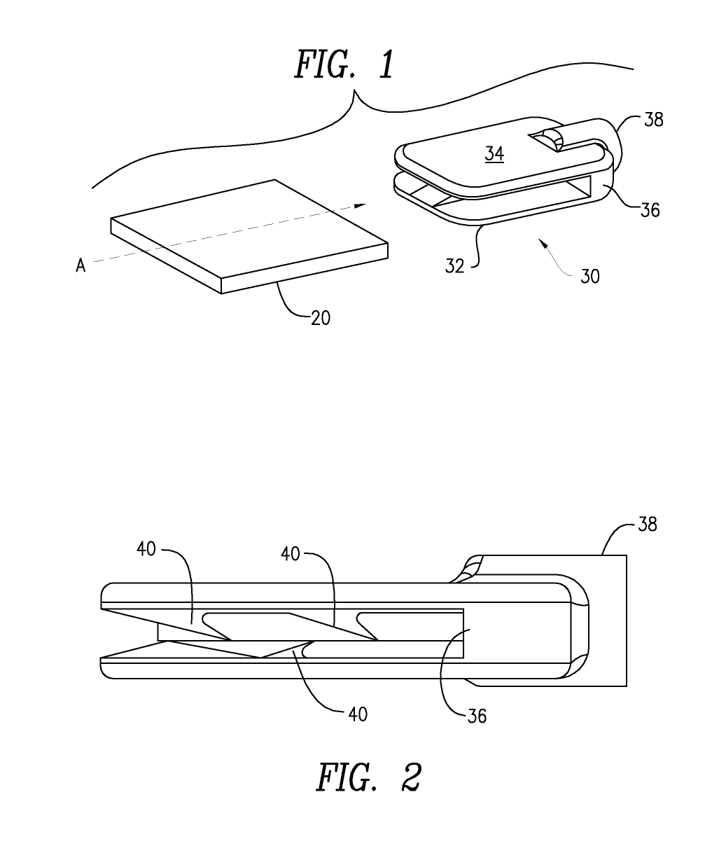

As shown in FIGS. 1 and 2, the user slides a standard alcohol pad 20 in the direction of arrow A into cover device 30. Cover device 30 can take on numerous configurations. As shown, cover device 30 includes opposing sides 32 and 34 joined at one end by end member 36, which can include housing member 38. The inner surfaces of sides 32 and 34 can have one or more teeth 40 that are angled toward end member 36 to allow one way entry of the prep pad 20. As such, the prep pad 20 can be inserted into cover device 30 by sliding the prep pad 20 into the open end of the cover device 30, between the sides 32 and 34, such that the teeth 40 cut open or pierce the prep pad package if the prep pad 20 is still in the package. If the pad 20 is removed from the package, it could be slid into the cover device 30 and the teeth 40 can retain the pad 20 in the cover device 30. The prep pad 20 can be exposed or put in contact with wicking material which absorbs disinfectant. The wicking material could include any suitable material, such as cellulose, polyurethane sponge, silicone sponge, porous plastic, bonded fiber, etc. A luer access device can be inserted into cover device 30 though a cavity or pocket or entry port end member 36 and housing member 38 to put the luer access device into contact with the disinfectant and to maintain the luer access device within cover device 30 until the next use of the luer access device. Housing member 38 can contain retention features to retain cover device on the luer access device. These features can include threads, snaps, protrusions, interference fits or other known attachment mechanisms.

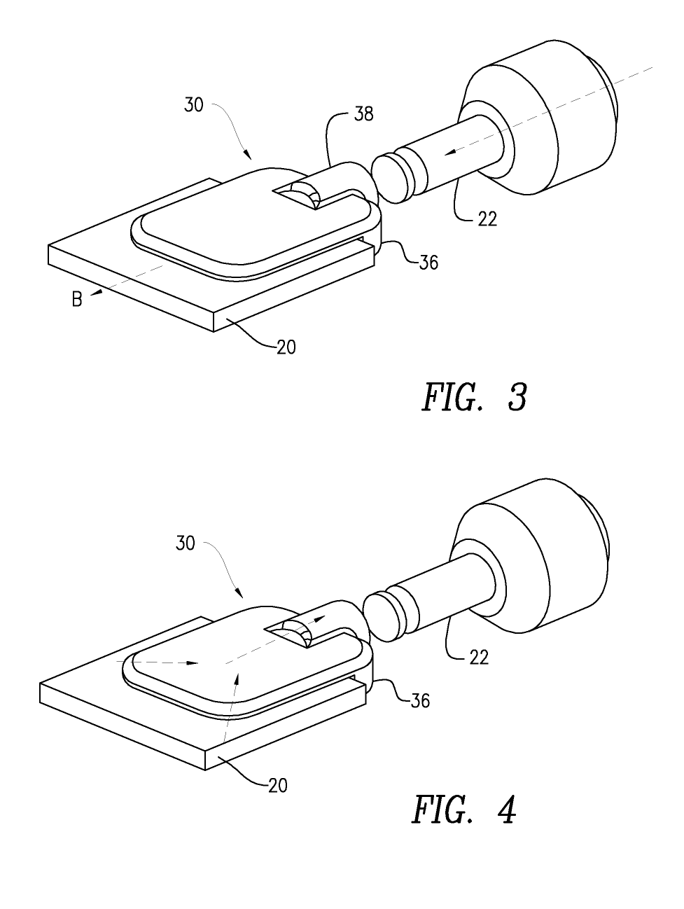

Referring to FIG. 3, it can be seen that the luer access device 22 can be inserted into end member 36 and housing member 38 of cover device 30. It can also be seen that after prep pad 20 is positioned between sides 32 and 34 of the cover device, it can pulled in the direction of arrow B to engage teeth 40 to pierce the packaging. As shown in FIG. 4, after the package is pierced, disinfectant can flow to the closed end member 36 where it can contact the luer access device 22.

Features that are pierceable or have pierceable access areas can be incorporated in the foil pouches to better enable predictable opening or fluid channels to be formed enabling the communication with an externally mounted device. The cover device could also be a self-adhered device. The user could stick the device to the package, press it or bend it to pop it open allowing the alcohol to wick into the wicking media and into the LAD cavity/recess.

In another aspect, the cover device could be formed as an extrusion. As such, a sponge could be positioned in an polypropylene (PP) or polyethylene (PE) extrusion and sealed inside a foil pack. The foil can be bent to ‘pop’ open a perforation or score and expose an opening in the sponge. The luer access device can be inserted into the sponge and the extrusion can spring back ‘gripping’ the device and holding the sponge with disinfectant, such as alcohol, in place on the luer access device.

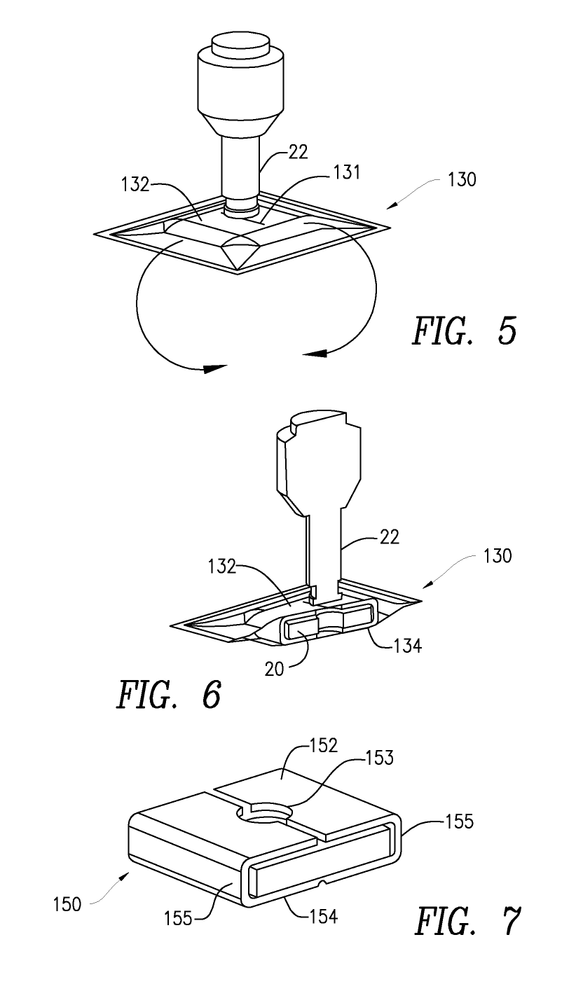

As shown in FIGS. 5-7, luer access device 22 can be inserted into cover device 130. Cover device 130 can be bent, curled, or otherwise manipulated to expose opening 131 through which luer access device 22 can be inserted. Opening 131 could also be initially covered by an additional adhesive lid, or sealing member that, once peeled away or punctured, could expose opening 131 for access by the luer access device 22. Upper and lower sides 132 and 134 can retain a pad 20 with disinfectant. The pad 20 can be included as part of subassembly 150 having an extruded perimeter with a top surface 152 that may be bifurcated and may have an opening 153, and a bottom surface 154, as well as sides 155. The cover device 130 can be comprised of foil which could be popped open or which could have a weakened area so that a luer access device can be popped through and into a pad with disinfectant. The pad or sponge can have a recess or hole so that it is donut shaped and can include a soft core positioned within the hole or recess. The cover device can spring back to engage or grip onto the luer access device to retain the cover device on the luer access device. The inner sponge can clean the tip of the luer access device while the donut shaped sponge can clean the outer surface. The device can be made and used without the soft core sponge, or it can be replaced with a thin pad or gauze to contact the tip of the luer access device.

In another aspect, an off-the-shelf alcohol prep pad can be used with a clip cover device to channel the alcohol into a luer type receptacle that helps to disinfect the surface of a luer access device. The clip cover device could have a clam-shell construction which receives a prep pad that a user drops into the device and which can then be closed on a luer access device. Alternatively, the clip cover device could snap onto a luer access device and then a prep pad can be dropped in and then the device can be closed.

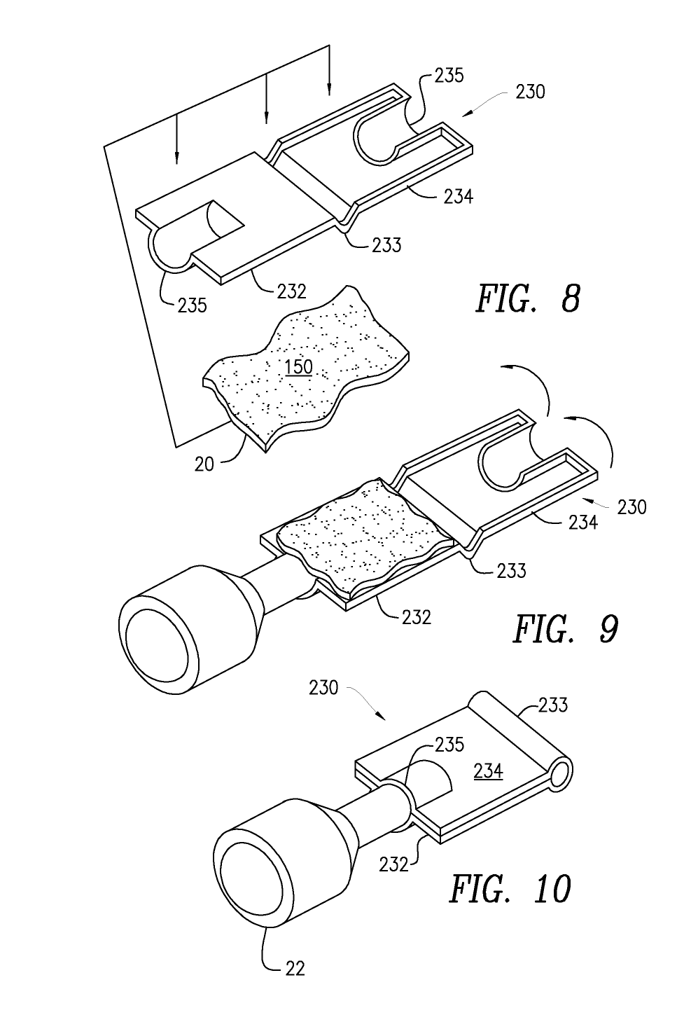

Referring to FIGS. 8-10, clip cover device is generally indicated at 230 and includes first and second sides 232 and 234 joined together such as by living hinge 233. A luer access receptacle 235 can be partially formed on each side 232 and 234 to form a luer access receptacle when the sides are brought together. Prep pad 20 can be positioned on one side, for example 232, and the other side, e.g. 234 can be closed/snapped down by pivotal movement about living hinge 233 in the direction of arrows C as shown in FIG. 9 to clamp the pad 20 between sides 232, 234. As shown in FIG. 10, a luer access valve 22 can be cleaned by insertion into the clip cover device 230 through luer access receptacle 235. Receptacle 235 can contain retention features to retain cover device on the luer access device. These features can include threads, snaps, protrusions, interference fits or other known attachment mechanisms.

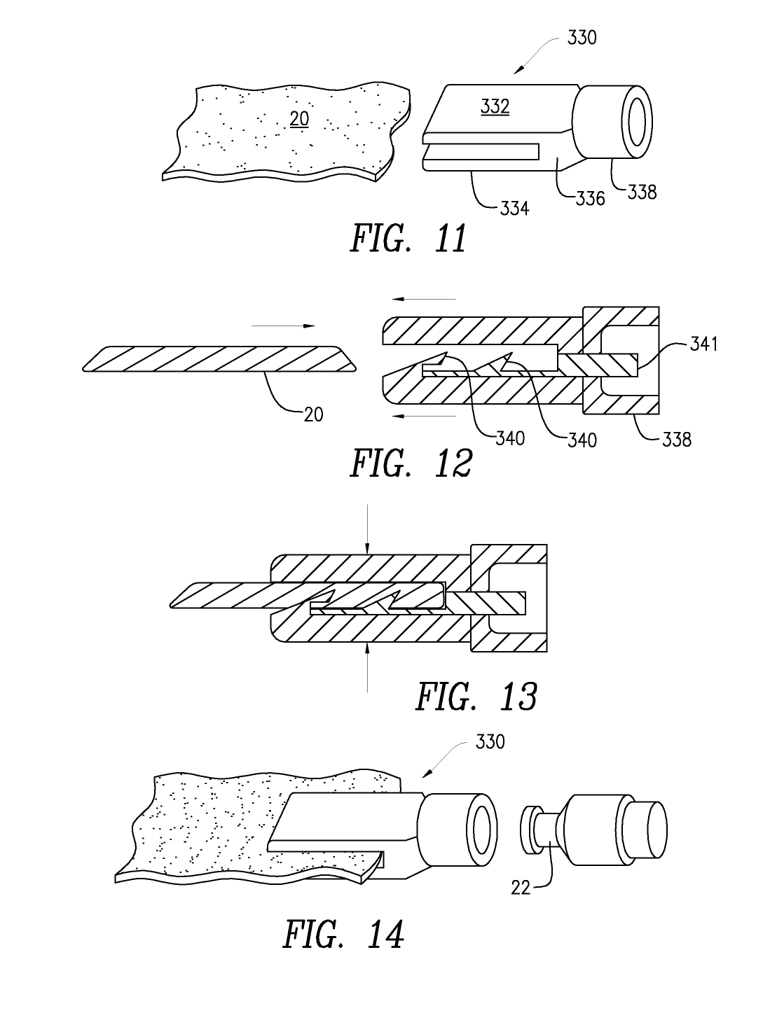

FIGS. 11-14, show another aspect wherein the user slides a standard alcohol pad 20 into cover device 330. Cover device 330 can take on numerous configurations. As shown, cover device 330 includes opposing sides 332 and 334 joined at one end by end member 336, which can include luer access port 338. One or both of inner surfaces of sides 332 and 334 can have one or more teeth 340 that are angled toward end member 336 to allow one way entry of the prep pad 20. As such, the prep pad 20 can be inserted into cover device 330 by sliding the prep pad 20 into the open end of the cover device 330, between the sides 332 and 334. After prep pad 20 is positioned between sides 332 and 334 of the cover device 330, it can pulled back to engage teeth 340 to pierce the packaging. The prep pad 20 can be exposed or put in contact with wicking material 341 which absorbs alcohol and delivers it to port 338. A luer access device 22 can be inserted into cover device 330 though port 338 in a slight interference snap fit to put the luer access device into contact with the alcohol to clean the luer access device and to maintain the luer access device within cover device 330 until the next use of the luer access device. Port 338 can contain retention features to retain cover device on the luer access device. These features can include threads, snaps, protrusions, interference fits or other known attachment mechanisms.

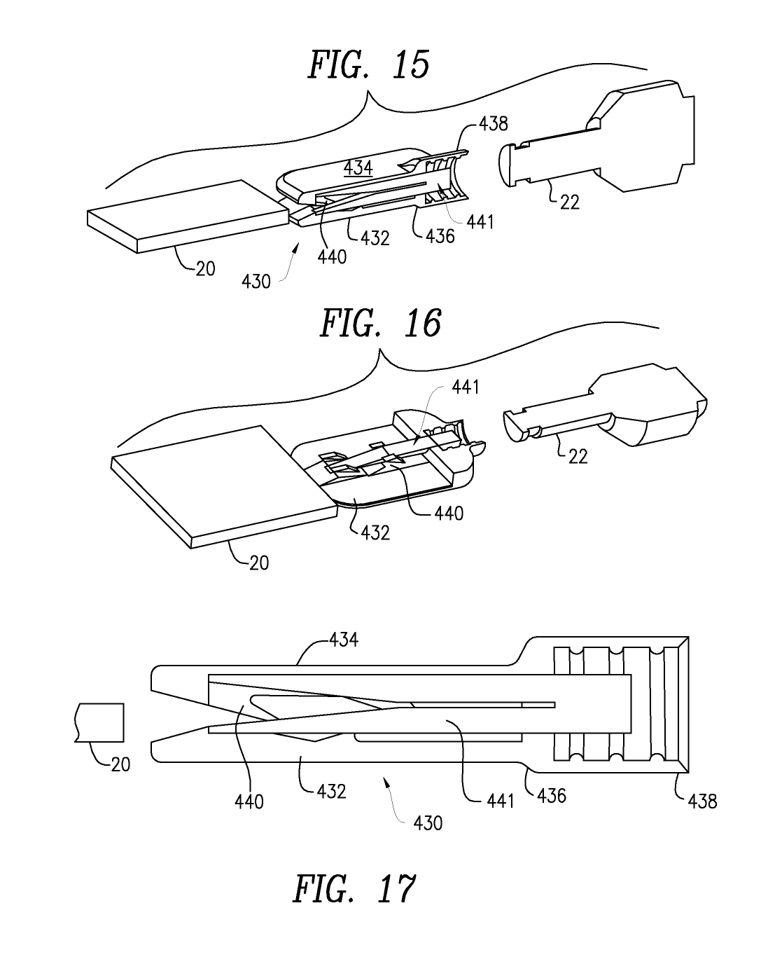

FIGS. 15-17 show another aspect of the cover device with a wicking material. Cover device 430 includes opposing sides 432 and 434 joined at one end by end member 436, which can include luer access port 438. One or both of inner surfaces of sides 432 and 434 can have one or more teeth 440 that are angled toward end member 436 to allow one way entry of the prep pad 20. As such, the prep pad 20 can be inserted into cover device 430 by sliding the prep pad 20 into the open end of the cover device 430, between the sides 432 and 434. After prep pad 20 is positioned between sides 432 and 434 of the cover device 430, it can pulled back to engage teeth 440 to pierce the packaging. The prep pad 20 can be exposed or put in contact with soft compressible wicking material 441 which absorbs alcohol and delivers it to port 438. The wicking material 441 could be split as shown to facilitate the entry of the prep pad 20. Alternatively, the wicking material 441 may be sufficiently offset to allow the prep pad 20 to slide on. The end of the wicking material 441 adjacent the port 438 compresses down flatter when the device that needs disinfecting is inserted. Isopropyl alcohol or any other disinfectant could be expelled from the wicking material 441 and baths an access device 22 in the cavity in the port 438. The cavity in the port 438 could be cylindrical and could include circumferential inference rings to maintain the cover device 430 on the access device 22. A luer access device 22 can be inserted into cover device 430 though port 438 to put the luer access device into contact with the alcohol to clean the luer access device and to maintain the luer access device within cover device 430 until the next use of the luer access device.

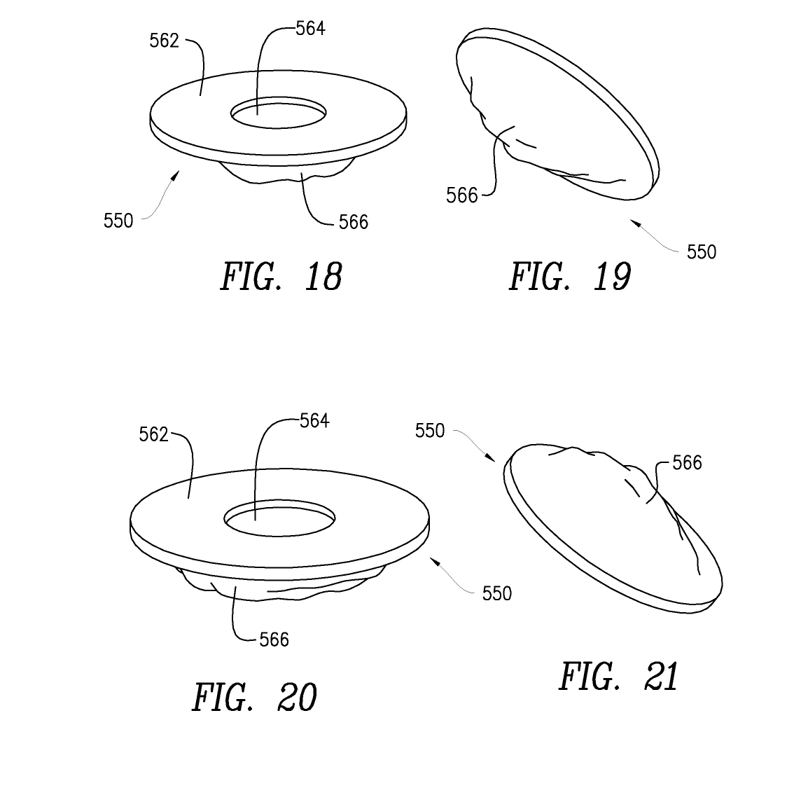

In another aspect, the cover device can have a punch through design that uses weaker foil or a breakable film or plastic area that allows a user to push a luer access device into an alcohol pad or sponge within the foil. The pad can have a recess or hole to accommodate the luer access device. FIGS. 18-21 show a cover device 550 having an upper surface 560 with an annular ring 562 and a central portion 564 sized to accommodate a luer access device. The central portion 564 can be pierced by insertion of a luer access device such that the tip of the luer access device extends into the cover device 550 and contacts a disinfecting pad therein for cleaning the luer access device. The central portion 564 can include any suitable material such as an aluminum foil or a thin plastic film to allow for a push or punch through design. The annular ring can be of any suitable shape and provides support for a breakable lidstock that can be pierced by the luer access device to access the central portion 564. Additionally, the annular ring 562 can be manufactured from a flexible material that engages protrutions, such as threads, on the luer access device upon insertion, to retain the cover on the luer access device. The back 566 of the cover device 550 can have any suitable construction such as a pouch made of any suitable material such as foil. The back 566 of the cover device 550 could be manufactured via a thermoforming process to create a “blister” cavity. Alternatively, the back 566 of the cover device 550 could be an injection molded housing made from any suitable material such as polypropylene or polyethylene. The backing material could be die cut, stiffer paper, metalized paper that is heated sealed, blow molded bottle, elastomeric sheet, or an injection molded cap. The cover device 550 may or may not include a lid 558. Alternatively, the luer access device 22 can be punched into the cover device 550 through the back 556. The inside of the back 566 could contain disinfectant itself, such as alcohol, or a pad or a sponge with disinfectant.

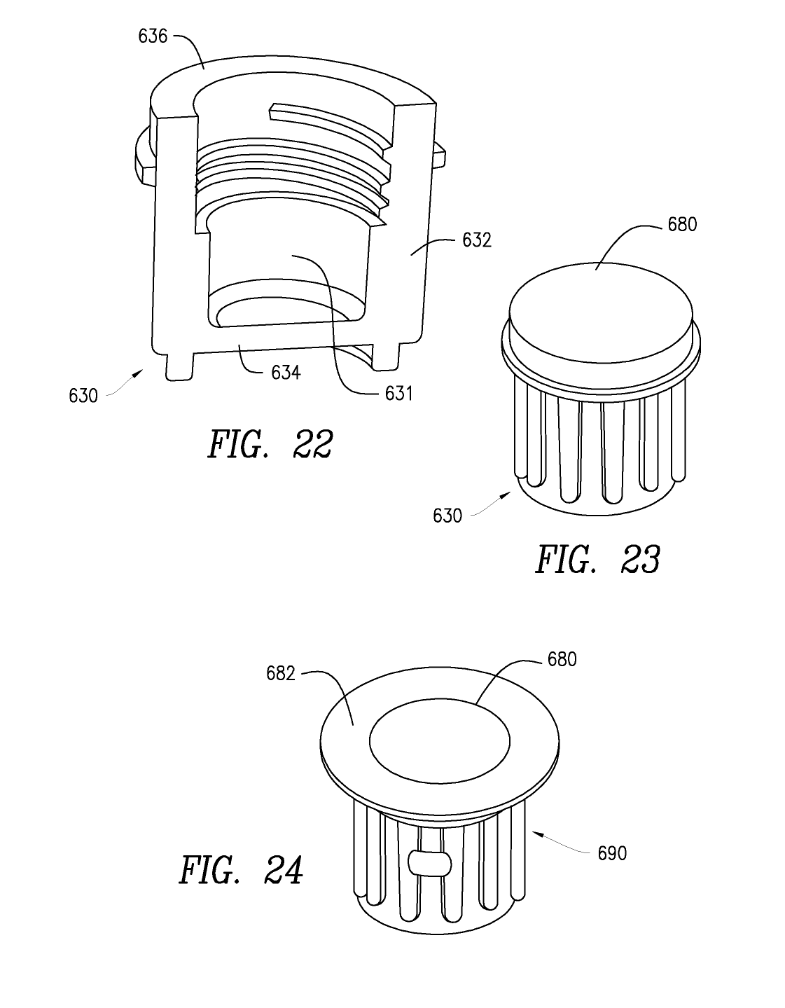

In another aspect, the cover device can take a cap structure as shown in FIGS. 22-24. A cap 630 includes a central chamber 631 defined by a side wall 632, a bottom 634 and an open top encircled by rim 636. A disinfectant is positioned within the chamber and punch through seal 680 is applied to the rim 636 at the upper edge of the cap 630. The punch through seal could include perforations or areas of weakness to insure it breaks where intended and that it does not become completely unattached (or that it does). The sealing material 680 can be applied to the cap using sealing materials and techniques to make it a non-removable seal. Such techniques could include using a film with a heat activated sealant layer that creates non peelable bond with the cap. Such adhesive seals could facilitate the punch through nature of the cap design. Other design features could further be incorporated into the cap to facilitate the punch through operation. For example a sharp edge or a serrated edge could be incorporated onto the opening of the cap, at the inner edge of rim 636. Furthermore the cap could incorporate features to control the fracture of the seal 680 to direct it in a specific direction, or to assure that it does not become completely unattached (or that it does). The inner surface of the sidewall 632 could be designed such that it mates very closely with the luer access valve upon fracture of the seal and insertion of the luer access device. Such a design could incorporate a slip fit. A slip fit, or similar design feature, would prevent the disinfectant from spilling out of the cap when the valve is inserted, in the case where the cap is applied upside down. The cap can incorporate threads that tighten to the luer access device and create a seal as the cap is applied to the luer access device to retain the disinfectant onto the luer access device for as long as possible. Longer retention times of the disinfectant could be associated with higher or faster levels of disinfection, as well as a longer period of protection from further contamination of the valve. In this aspect, the cap 630 does not include a sponge or silicone septum. The cap 630 could be provided with only a disinfectant, such as alcohol, and a punch through seal.

The inner surface of the upper portion of the disinfectant cap can include variable pitch threads. The threads can start out at a constant pitch (e.g., starting pitch), such as for an approximate ¼-¾ of a turn, and then change pitch thereafter. The starting pitch is that of a standard luer lock design to assist the user with applying the disinfectant cap to an LAD (e.g., relatively low torque). By varying the pitch (e.g., by reducing or increasing the pitch), as the disinfectant cap is threaded onto the LAD (or other medical device), the consistent standard threads of the medical device wedge with the mismatched variable pitch threads of the disinfectant cap.

Wedging the threads together in this fashion provides a more secured connection (e.g., from unthreading or loosening from incidental handling or contact) because a higher removal torque is needed to disengage the disinfectant cap from the LAD. To create and maintain the wedge, the disinfectant cap could be made of a variety of materials, such as a hard plastic (e.g., high-density polyethylene (HDPE)). LADs usually have ACME profiled threads or modified ACME profiled threads (e.g., stub ACME threads), which are known for power transmission applications due to the flat sides which distribute stress well over the faces of the thread. As a result, the ACME type threads transmit high torques while minimizing stress, which translates to better wedging action and higher removal torques. As such, the threads can seal the cap with the luer access device and prevent ingress of contaminates.

The tightness of fit can be enhanced by adding a reducing taper to the major inner diameter and/or minor inner diameter of the threads. Flexibility of the cap walls of the housing facilitates removal of the cap from a mold without the need for unscrewing due to the binding nature of the thread.

As shown in FIG. 24, the cap could be positioned within a cover 690 and the punch though seal 680 could be applied to the cover 690. A perforated ring 682, or other features, could be employed to insure the seal 680 breaks where intended.

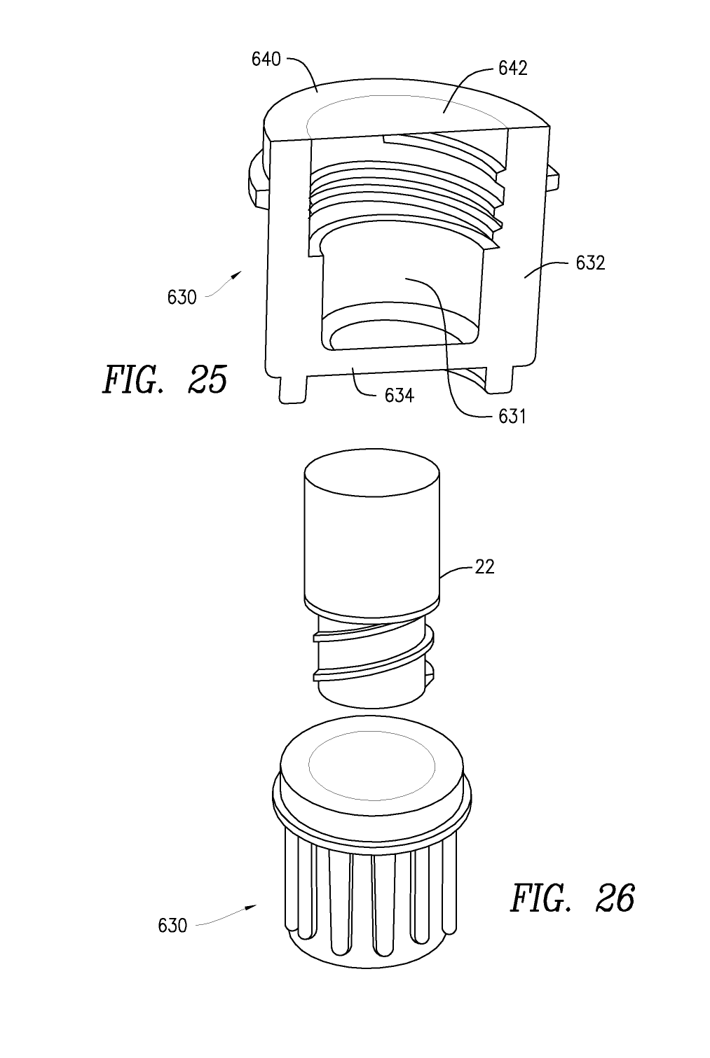

FIG. 25 is a cross-sectional view of the cap punch through seal shown in FIG. 23. The cap includes a non-peelable seal 640 and a push through area 642.

FIG. 26 shows a luer access device positioned to be pushed into the cover. A relationship exists between the outer diameter of the valve and the inner diameter of the cap such that the minimal space provided retains the disinfectant during initial valve insertion.

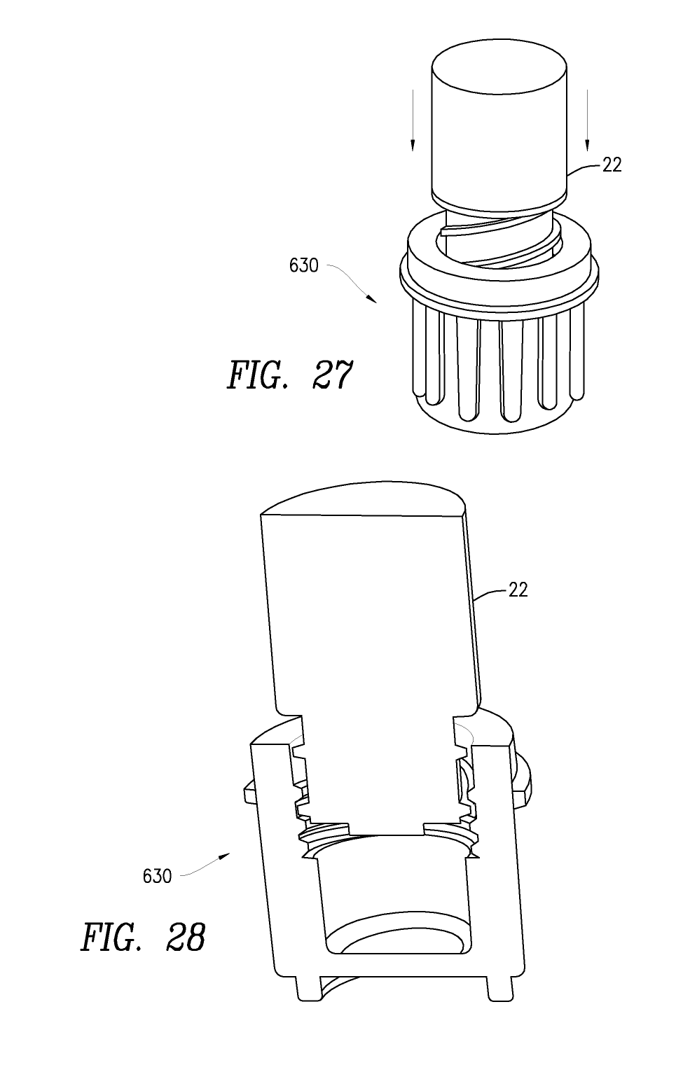

FIG. 27 shows a luer access device pushed into the cover.

FIG. 28 shows a luer access device fully seated in the cover.

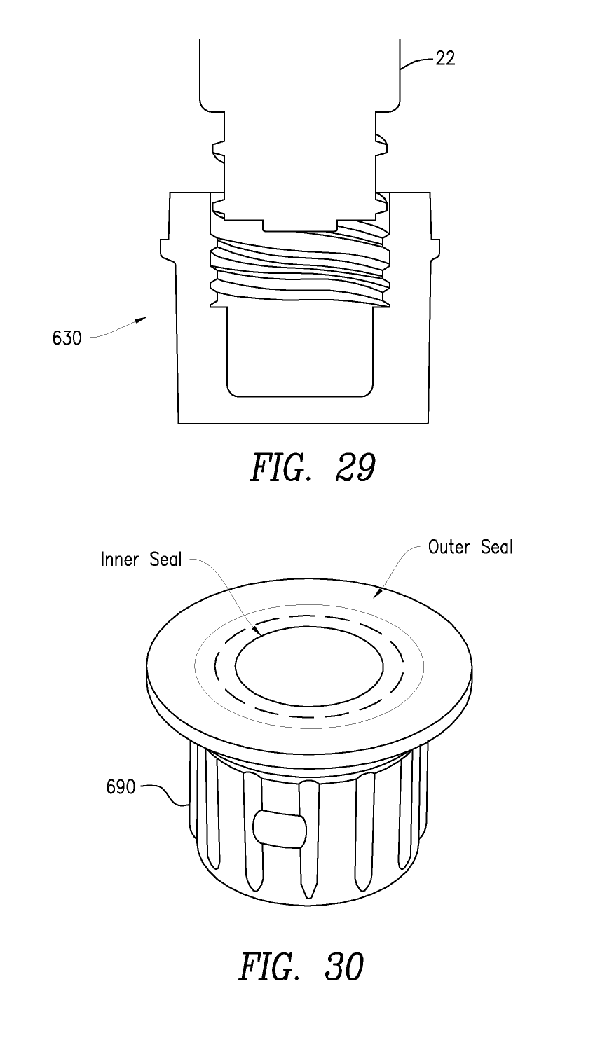

FIG. 29 is a cross-sectional view of the luer access device and cover as shown in FIG. 27. Small space outer diameter of valve and inner diameter of cap that facilitates disinfectant pool to be retained inside of cap is shown at 29A.

FIG. 30 is a more detailed view of the holder shown in FIG. 24. The following push through barrier has two seal areas. The inner seal area is a non-peelable seal. The outer seal may be either peelable or non-peelable. The perforated or scored area allows the LAD to be removed from the holder.

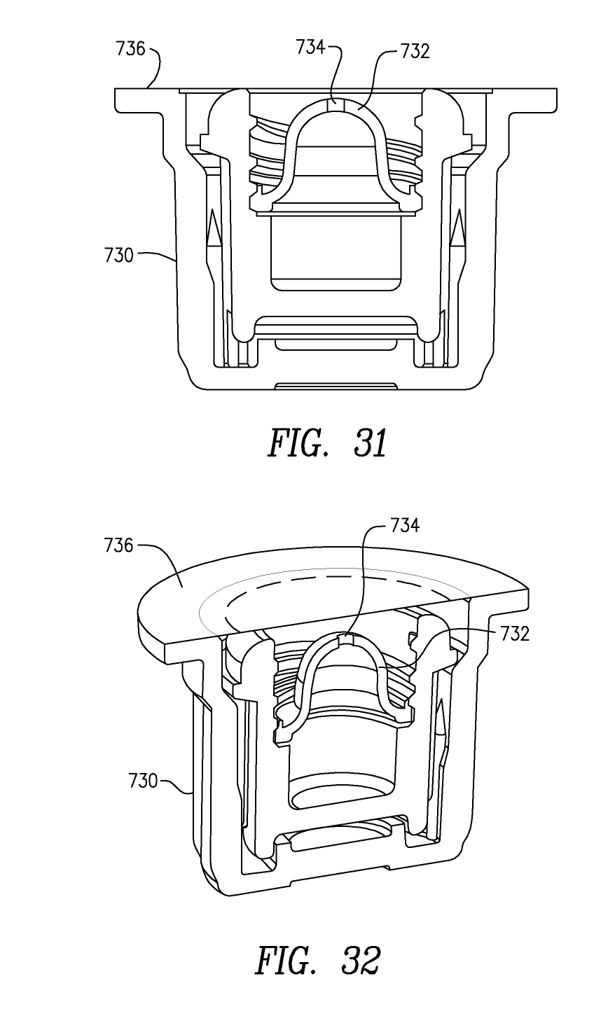

FIG. 31 shows a cap 730 with a cover 736 and a flexible septum 732 with an aperture 734 that retains a disinfectant such as alcohol within a chamber formed by the inside of the cap 730 and the septum 732.

FIG. 32 is a perspective view of the body shown in FIG. 31.

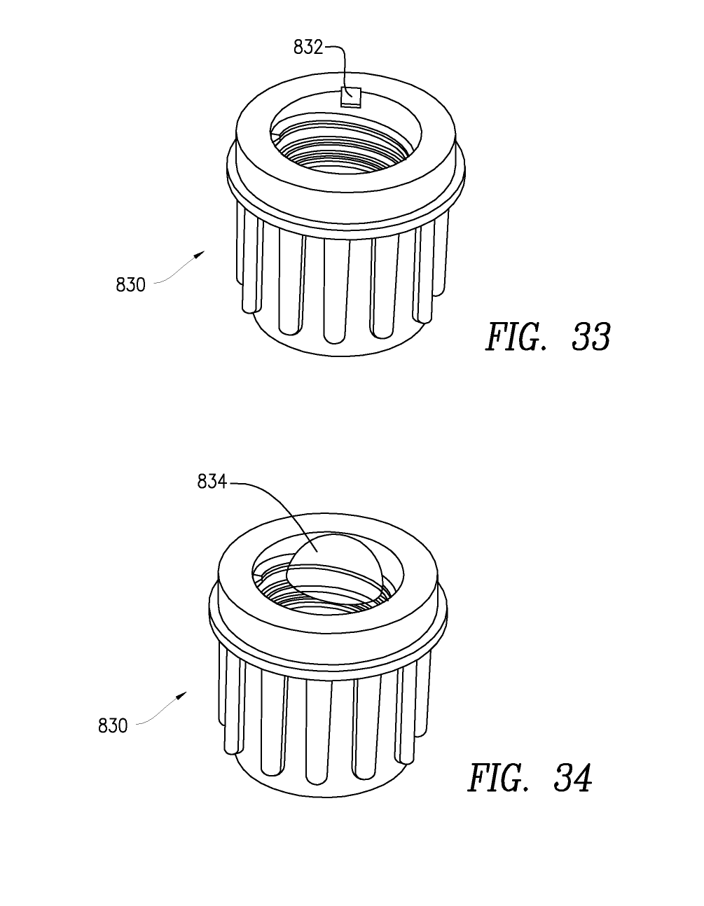

FIG. 33 shows a cap 830 with a recess 832 or notch molded in the inner surface of the sidewall. The recess 832 retains a punch through flap 834 shown in FIG. 34 or seal after valve insertion so that the punch through flap does not become completely unattached. The recess 832 is designed so that the punch through flap does not break in that area. Other mechanisms could be employed to retain the punch through flap, such as including a plurality of recesses around the circumference of the cap.

FIG. 34 is a perspective view of the cap 830 with a punch through flap 834 that remains attached to the cap 830 after use. The punch through flap 834 remains attached to the recess 832 shown in FIG. 33 after a valve has been inserted.

While the invention has been described in detail and with reference to specific examples thereof, it will be apparent to one skilled in the art that various changes and modifications can be made therein without departing from the spirit and scope thereof.

What is claimed is:

1. A device for cleaning access devices, comprising:
a body with a first opposing side, a second opposing side, and a closed end, the body defining a space for retaining a disinfectant; and
a housing configured to engage an access device.

2. The device of claim 1, wherein the body has a protrusion angled toward the closed end to allow one way entry of the pad into the body.

3. The device of claim 2, wherein the protrusion includes teeth to pierce the pad to expose the disinfectant.

4. The device of claim 3, wherein the body includes a wicking material for absorbing the disinfectant.

5. The device of claim 4, wherein the wicking material includes cellulose.

6. The device of claim 1, wherein the housing is positioned at the closed end of the body.

7. The device of claim 1, wherein the body includes a bifurcated top surface and an opening for receiving the access device.

8. The device of claim 1, further comprising a living hinge connecting the first opposing side to the second opposing side.

9. The device of claim 8, wherein the housing is formed when the first opposing side is adjacent the second opposing side.

10. The device of claim 9, wherein the first opposing side and the second opposing side cooperate to form the pocket when the first opposing side is adjacent the second opposing side.

11. A device for cleaning access devices, comprising:
a body including an upper surface and a lower surface, the body including a disinfecting pad, the upper surface including an annular ring and a central portion being pierceable by insertion of a luer access device, the lower surface including a pouch.

12. The device of claim 11, wherein the central portion includes aluminum foil.

13. An antiseptic cap for use with an access site, comprising:
a housing with a generally cylindrical sidewall defining a chamber having an open upper end, a rim formed at the open upper end, and an end wall closing an opposite end;
a disinfectant positioned within the chamber; and
a punch through seal applied at the rim.

14. The antiseptic cap of claim 13, wherein the punch through seal includes a perforated ring.

15. The antiseptic cap of claim 14, wherein the housing includes a recess for retaining the punch through seal.
